(19) United States
(12) Patent Application Publication
(10) Pub. No.: US 2012/0179038 A1
(43) Pub. Date: Jul. 12, 2012

(54) ULTRASOUND BASED FREEHAND INVASIVE DEVICE POSITIONING SYSTEM AND METHOD

(75) Inventors: Robert Andrew Meurer, Waukesha, WI (US); Menachem Halmann, Bayside, WI (US); Emil Markov Georgiev, Hartland, WI (US); Erik Paul Kemper, Franklin, WI (US); Jeffery Scott Peiffer, Waukesha, WI (US)

(73) Assignee: GENERAL ELECTRIC COMPANY, Schenectady, NY (US)

(21) Appl. No.: 12/986,753

(22) Filed: Jan. 7, 2011

(51) Int. Cl. A61B 8/14

(52) U.S. Cl. 600/443

(57) ABSTRACT

In one embodiment, an interventional guidance method includes generating an ultrasound image of a subject anatomy of interest. The method also includes superimposing on the ultrasound image a visual indication of at least one of projection of a position of an interventional device, trajectory of the interventional device, and a location at which the interventional device will intercept an ultrasound imaging plane. The interventional guidance method also includes dynamically altering an aspect of the superimposed visual indication during a interventional procedure. The dynamic altering includes altering a dynamic indication of a trajectory of the interventional instrument transverse to the imaging plane or an interception location of the trajectory of the interventional instrument with the imaging plane.

### BACKGROUND OF THE INVENTION

The subject matter disclosed herein relates to ultrasound systems, and, more particularly, to an ultrasound based freehand invasive device positioning system and method.

Ultrasound systems may be used to examine and study anatomical structures, and to assist operators, typically radiologist and surgeons, in performing medical procedures. These systems typically include ultrasound scanning devices, such as ultrasound probes, that transmit pulses of ultrasound waves into the body. Acoustic echo signals are generated at interfaces in the body in response to these waves. These echo signals are received by the ultrasound probe and transformed into electrical signals that are used to produce an image of the body part under examination. This image may be displayed on a display device.

When an ultrasound system is used to assist an operator in performing a medical procedure, the operator may hold an ultrasound probe in one hand, while holding a medical instrument in their other hand. The ultrasound image produced may include a representation of the medical instrument superimposed over the ultrasound image to assist the operator to correctly position the medical instrument. Unfortunately, the ultrasound image with the overlaid medical instrument representation may be a two dimensional figure that provides no actual indication of depth trajectory that may allow for enhanced three dimensional accuracy in the placement of the interventional instrument. Therefore, a system that enables an operator to receive feedback based on all three dimensions may increase the ability of an operator to rely on an ultrasound system while performing a medical procedure, thereby decreasing complications and improving controllability of the procedure.

### BRIEF DESCRIPTION OF THE INVENTION

In one embodiment, an interventional guidance method includes generating an ultrasound image of a subject anatomy of interest. The method also includes superimposing on the ultrasound image a visual indication of at least one of projection of a position of an interventional device, trajectory of the interventional device, and a location at which the interventional device will intercept an ultrasound imaging plane. The interventional guidance method also includes dynamically altering an aspect of the superimposed visual indication during an interventional procedure. The dynamic altering includes altering a dynamic indication of a trajectory of the interventional instrument transverse to the imaging plane or an interception location of the trajectory of the interventional instrument with the imaging plane.

In another embodiment, an interventional guidance system includes an ultrasound system configured to generate an ultrasound image of a subject anatomy of interest and a display. The display is configured to show the ultrasound image and a visual indication of at least one of projection of a position of an interventional device, trajectory of the interventional device, and a location at which the interventional device will intercept an ultrasound imaging plane. The interventional guidance system also includes the visual indication superimposed on the ultrasound image. The system includes an aspect of the superimposed visual indication that is dynamically altered during an interventional procedure. The dynamic altering includes altering a dynamic indication of a trajectory of the interventional instrument transverse to the imaging plane or an interception location of the trajectory of the interventional instrument with the imaging plane.

In a further embodiment, an interventional guidance method includes generating an ultrasound image of a subject anatomy of interest. The method also includes superimposing on the ultrasound image a visual indication of at least one of projection of a position of an interventional device, trajectory of the interventional device, and a location at which the interventional device will intercept an ultrasound imaging plane. The interventional guidance method also includes providing auditory feedback indicative of at least one of proximity of the interventional device to the subject anatomy of interest, and a degree of correctness or error of a current trajectory of the interventional device to the subject anatomy of interest.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an ultrasound system in accordance with aspects of the present disclosure;

FIG. 2 is a flow chart of a method of interventional instrument positioning employing the ultrasound system of FIG. 1;

FIG. 3 is a perspective view of an in-plane navigation display of the ultrasound system of FIG. 1 illustrating the interventional instrument on target;

FIG. 4 is a perspective view of an in-plane navigation display of the ultrasound system of FIG. 1 illustrating the interventional instrument behind the target;

FIG. 5 is a perspective view of an in-plane navigation display of the ultrasound system of FIG. 1 illustrating the interventional instrument ahead of the target;

FIG. 6 is a perspective view of an out of plane navigation display of the ultrasound system of FIG. 1 illustrating the interventional instrument on target;

FIG. 7 is a perspective view of an out of plane navigation display of the ultrasound system of FIG. 1 illustrating the interventional instrument ahead of the target; and

FIG. 8 is a perspective view of an out of plane navigation display of the ultrasound system of FIG. 1 illustrating the interventional instrument behind the target.

### DETAILED DESCRIPTION OF THE INVENTION

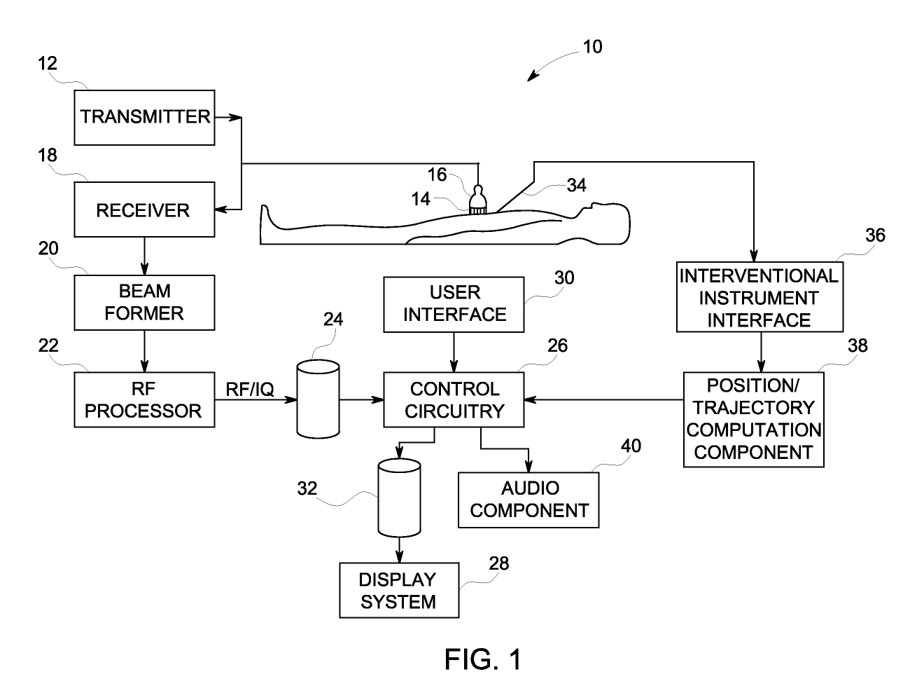

FIG. 1 is a block diagram of an ultrasound system 10 that may be used, for example, to acquire and process ultrasonic images. The ultrasound system 10 includes a transmitter 12 that drives one or more arrays of elements 14 (e.g., piezoelectric crystals) within or formed as part of a probe 16 to emit pulsed ultrasonic signals into a body or volume. A variety of geometries may be used and one or more transducers may be provided as part of the probe 16. The pulsed ultrasonic signals are back-scattered from density interfaces and/or structures, for example, in a body, like blood cells or muscular tissue, to produce echoes that return to the elements 14. The echoes are received by a receiver 18 and provided to a beam former 20. The beam former 20 performs beamforming on the received echoes and outputs an RF signal. The RF signal is then processed by an RF processor 22. The RF processor 22 may include a complex demodulator (not shown) that demodulates the RF signal to form IQ data pairs representative of the echo signals. The RF or IQ signal data then may be routed directly to an RF/IQ buffer 24 for storage (e.g., temporary storage).

The ultrasound system 10 also includes control circuitry 26 to process the acquired ultrasound information (i.e., RF signal data or IQ data pairs) and to prepare frames of ultrasound information for display on a display system 28. The control circuitry 26 may be adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 24 during a scanning session and processed in less than real-time in a live or off-line operation.

The display system 28 may include a display screen, such as a navigation display, to display the ultrasound information. A user interface 30 may be used to control operation of the ultrasound system 10. The user interface 30 may be any suitable device for receiving user inputs to control, for example, the type of scan or type of transducer to be used in a scan. As such, the user interface may include a keyboard, mouse, and/or touch screen, among others.

The ultrasound system 10 may continuously acquire ultrasound information at a desired frame rate, such as rates exceeding fifty frames per second, which is the approximate perception rate of the human eye. The acquired ultrasound information may be displayed on the display system 28 at a slower frame rate. An image buffer 32 may be included for storing processed frames of acquired ultrasound information that are not scheduled to be displayed immediately. In one embodiment, the image buffer 32 is of sufficient capacity to store at least several seconds of frames of ultrasound information. The frames of ultrasound information may be stored in a manner to facilitate retrieval thereof according to their order or time of acquisition. The image buffer 32 may comprise any known data storage medium.

An interventional instrument 34 may be used as part of the ultrasound system 10 to enable a user to perform a medical procedure on a patient while collecting ultrasound information from the probe 16. The interventional instrument 34 may be a needle, catheter, syringe, cannula, probe, or other instrument and may include sensors, gyroscopes, and/or accelerometers to aid in determining position information of the interventional instrument 34. An interventional instrument interface 36 may receive electrical signals from the interventional instrument 34 and converts these signals into information such as position data, orientation data, trajectory data, or other sensor information. A position/trajectory computation component 38 may calculate the orientation and physical location of the interventional instrument 34 using the information from the interventional instrument interface 36. The control circuitry 26 may receive the interventional instrument 34 location and orientation data and prepares the information to be shown on the display system 28. The control circuitry 26 may cause an ultrasound image and an image or representation of the interventional instrument 34 to be overlaid when depicted on the display system 28, along with target locations, plane intercept points, trajectories, and so forth, as described below. Furthermore, an audio component 40 may be used to give audible information about the location and/or orientation of the interventional instrument 34 to an operator.

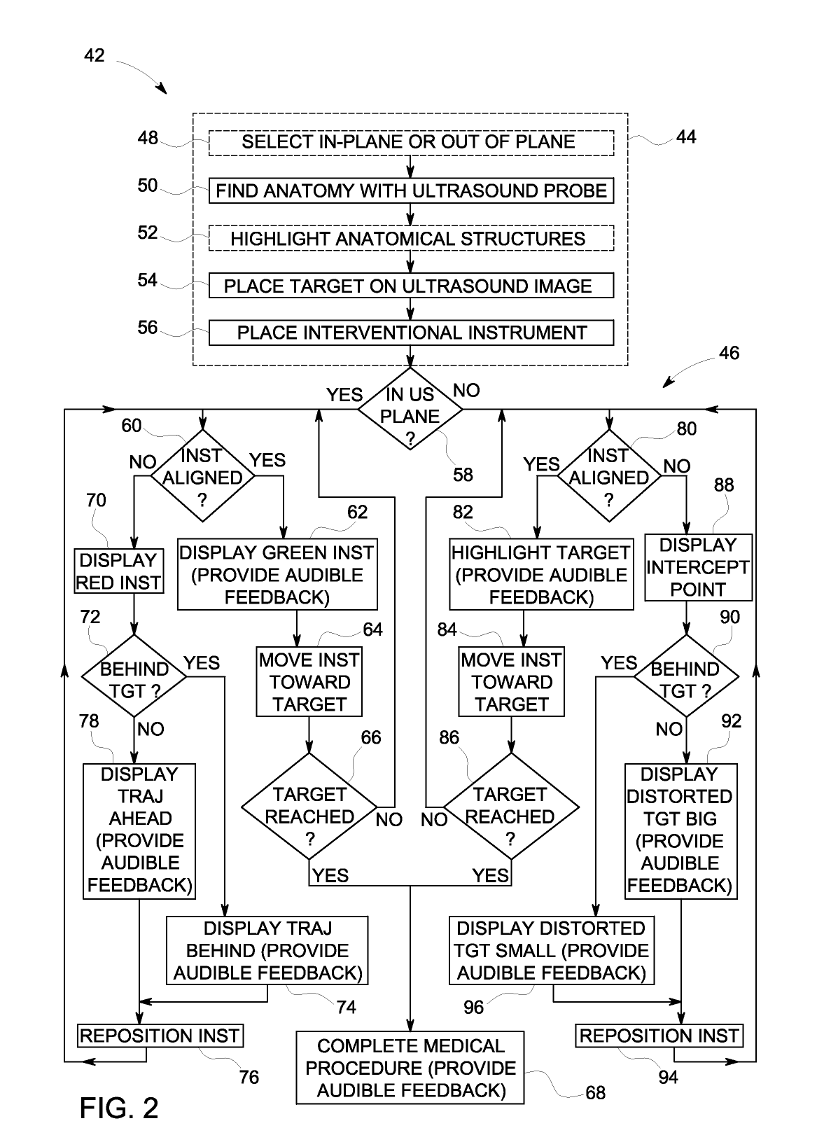

FIG. 2 is a flow chart of a method of interventional instrument guidance 42 employing the ultrasound system of FIG. 1, while the remaining figures represent exemplary displays of ultrasound images with interventional instrument trajectories, intercept points, and so forth, as described below with reference to the flow chart of FIG. 2. Preparation steps 44 may be performed prior to a navigation procedure 46. Other embodiments, however, may not include the preparation steps 44. Furthermore, not all steps described are necessary for interventional instrument guidance and the steps may be performed in an order other than as described.

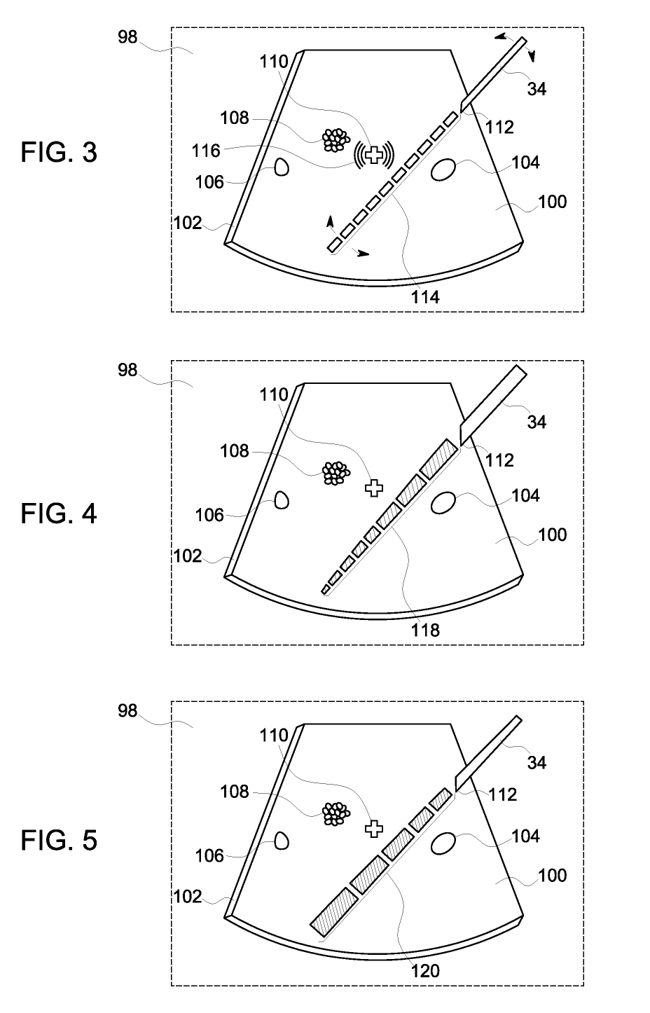

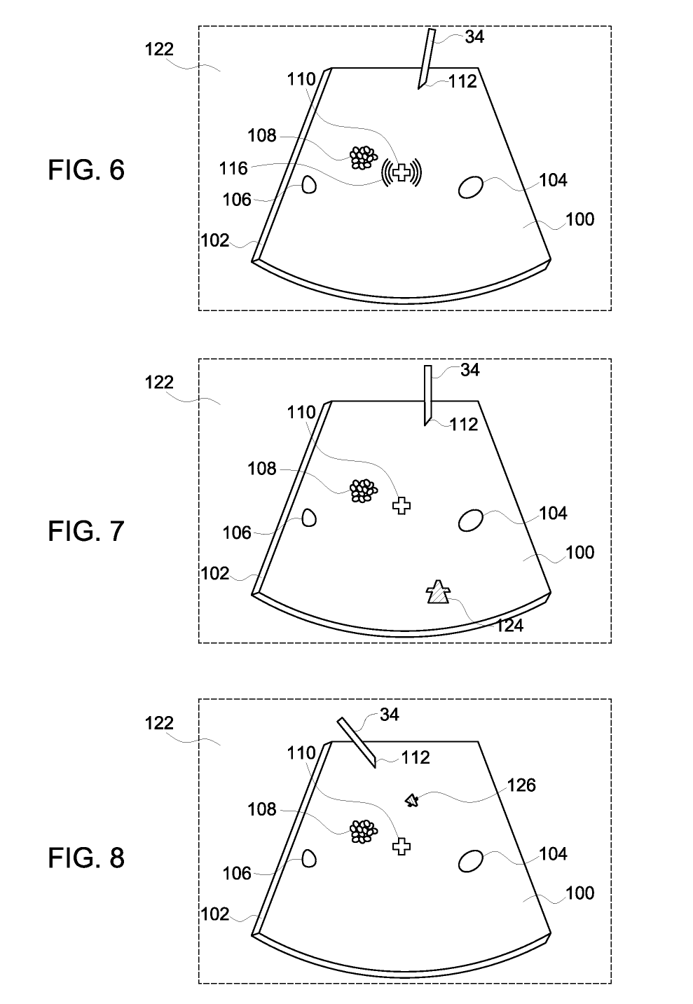

The preparation steps 44 may include step 48 where in-plane or out of plane navigation is selected. The result of the selection of in-plane or out of plane navigation may be used to determining the manner of displaying the interventional instrument. For example, FIGS. 3 through 5 depict exemplary navigational aid screens that may appear as part of an in-plane navigation display 98, while FIGS. 6 through 8 depict similar exemplary navigational aid screens for an out of plane navigation display 122. In-plane or out of plane navigation may be selected manually by a user making the selection, or the selection may be performed automatically such as by the position/trajectory computation component and/or the control circuitry. Furthermore, the control circuitry may use the position and orientation information acquired from the interventional instrument and the ultrasound probe to determine whether the interventional instrument is in or out of the ultrasound plane. As used in the present discussion, the term “in-plane” refers to a procedure in which an instrument is inserted and advances towards a target point with a trajectory that lies generally within a plane of imaging by the ultrasound system. The term “out of plane”, on the other hand, refers to a procedure in which the instrument originates (i.e., is initially inserted into the patient) out of the imaging plane, but advances into or traverses the imaging plane along its desired trajectory.

At step 50, a user may find an anatomy of interest on the subject using the ultrasound probe. For example, the user may perform a procedure involving the appendix and may move the ultrasound probe over the body of the subject until the display system shows the appendix within the acquired ultrasound image. When the anatomy of interest is located, the user may highlight certain anatomical structures on the display showing the ultrasound image per step 52. The user may highlight the anatomical structures by providing input from the user interface to cause anatomical structures to be displayed on the ultrasound image with a certain color, label, or bold outline, for example, or simply to place a viewable indictor on, around or near the anatomy. Any anatomical structures may be highlighted, such as organs, arteries, veins, specific tissues or part of tissues, nerve bundles, and so forth. For example, in FIGS. 3 through 8, an ultrasound image 100 is illustrated having an ultrasound plane 102. Within the ultrasound plane 102, an artery 104, a vein 106, and a nerve bundle 108 are depicted and may be highlighted to enable the user to easily see the anatomical structures during the interventional procedure. Highlighted anatomy may enable the user to move the interventional instrument 34 to avoid contact with the anatomy during the interventional method 42.

Returning to FIG. 2, the user may place a target on the ultrasound image at step 54. Again, FIGS. 3 through 8 illustrate how a target 110 may be depicted on the ultrasound image 100. Although the target 110 is depicted as a cross, other embodiments may depict the target 110 as a circle, square, oval, triangle, or another shape useful to designate a target. Continuing on to step 56, a user may place the interventional instrument at a desired location on the subject. For example, FIGS. 3 through 8 illustrate an interventional instrument 34 with a tip 112 displayed over the ultrasound image 100. Returning to step 56, if the ultrasound probe is rotated approximately 180 degrees from a proper orientation in relation to the interventional instrument, a caution statement may be displayed on the navigation display informing the user that the probe needs to be rotated 180 degrees for proper orientation.

At step 58, the prior selection of in-plane or out of plane navigation may be used to determine whether the interventional instrument is in the ultrasound plane. Alternatively, the ultrasound system may automatically determine whether the interventional instrument is in-plane or out of plane. If the interventional instrument is in the ultrasound plane, the control circuitry may determine whether the interventional instrument is aligned to intercept the target per step 60. If the interventional instrument is aligned properly, the interventional instrument and/or its projected path may be displayed on the navigation display with a green color at step 62. For example, FIG. 3 illustrates a projected path 114 of the interventional instrument 34. The projected path 114 and/or the interventional instrument 34 may be displayed in a desired way, such as in a specific color (e.g., green color) if the interventional instrument 34 is aligned with the target 110. However, in other embodiments colors other than green may be used to depict alignment with the target 110, such as yellow, purple, or orange, or indeed any useful graphical indicia may be used to provide similar indications. Furthermore, the projected path 114 and/or the interventional instrument 34 may be displayed with a generally uniform width, as illustrated, when aligned in two of three dimensions. Although the interventional instrument 34 has a projected path 114 that is in-plane (first dimension alignment) and is aligned in a second direction, the interventional instrument 34 may be positioned in a third direction as depicted by the arrows to be aligned with the target 110 in all three dimensions. When the interventional instrument 34 is aligned in all three dimensions, the projected path 114 may be depicted as extending to or through the target 110. An alignment indicator 116 may be displayed to further illustrate that the interventional instrument 34 is aligned with the target 110.

Returning to FIG. 2, step 62 may also include providing audible feedback. The audible feedback may be an additional feature to provide a user with information about the interventional instrument guidance. For example, the ultrasound system may provide the user with a pulsed audible tone at a frequency within a normal human auditory range, such as between 85 and 255 Hz, when the interventional instrument is in the ultrasound plane and aligned. The time between the audible tone pulses may decrease as the interventional instrument approaches to the target. Similarly, the pitch (frequency) of the audible feedback may change, such as depending upon whether the trajectory would intercept the target or not (with frequencies changing as the trajectory moves towards or away from the target). At step 64, the user may move the interventional instrument toward the target. Next, at step 66, the control circuitry may determine whether the target is reached. If the target is not reached, the interventional method 42 returns to step 60. If the target is reached, the user may complete the medical procedure per step 68. In addition, when the target is reached the ultrasound system may provide audible feedback. For example, the ultrasound system may continue to provide the user with auditory feedback, such as an uninterrupted audible tone at a frequency within the normal human auditory range when the target is reached.

If the interventional instrument is not aligned at step 60, the interventional instrument and/or the projected path of the interventional instrument may be displayed in a different manner, such as in red per step 70. Alternatively, other embodiments may use orange, blue, white, black, or any other color, or indeed any perceptible graphical presentation that may be used to assist a user in differentiating between whether the interventional instrument is aligned or not aligned with the target. At step 72, the control circuitry may determine whether the interventional instrument is heading behind the target. If the interventional instrument is headed behind the target, the projected path of the interventional instrument may be displayed on the navigation display as if the interventional instrument were heading behind the target per step 74. For example, FIG. 4 depicts the interventional instrument 34 with its projected path 118 diminishing in size as the projected path 118 extends into the ultrasound image 100. With the projected path 118 diminishing in size, the view on the navigation display 98 makes it appear that the interventional instrument 34 is heading behind the target 110. In addition, the interventional instrument 34 and/or the projected path 118 may be portrayed in a manner different than that used when the interventional instrument 34 is aligned with the target 110 (e.g., in a different color). For example, if the color is green when the interventional instrument 34 is aligned, the color may be red when the interventional instrument 34 is not aligned. Furthermore, the color of the interventional instrument 34 and/or projected path 118 may transition through various color shades as the interventional instrument 34 and/or projected path 118 get closer to or further away from the target 110.

Returning to FIG. 2, at step 74 the ultrasound system may provide audible feedback to the user to assist the user in positioning the interventional instrument. For example, here again the ultrasound system may provide the user with an uninterrupted audible tone below the range of normal human auditory frequencies when the interventional instrument is heading behind the target, such as below 85 Hz. Furthermore, the frequency may be adjusted higher or lower as the position of the interventional instrument moves respectively closer or further away from alignment with the target. Next, the user may reposition the interventional instrument at step 76, then return to step 60 where the steps may be repeated until the target is reached and the medical procedure is complete.

Resuming the method at step 72, if the control circuitry determines that the interventional instrument is not heading behind the target, the projected path of the interventional instrument and/or the interventional instrument may be portrayed on the navigation display as being ahead of the target per step 78. For example, FIG. 5 depicts the interventional instrument 34 and its projected path 120 increasing in size as the projected path 120 extends further into the ultrasound image 100. With the projected path 120 increasing in size, the view on the navigation display 98 makes it appear that the interventional instrument 34 is heading in front of the target 110.

Returning to FIG. 2, at step 78 the ultrasound system may again provide audible feedback to the user to assist the user in positioning the interventional instrument. For example, the ultrasound system may provide the user with an uninterrupted audible tone above the range of normal human auditory frequencies when the interventional instrument is heading in front of the target, such as above 255 Hz. Furthermore, the frequency may adjust lower or higher as the position of the interventional instrument moves respectively closer or further away from alignment with the target. Again, the user may reposition the interventional instrument at step 76, then return to step 60 where the steps may be repeated until the target is reached and the medical procedure is complete.

Resuming the method at step 58 in FIG. 2, if the interventional probe is not in the ultrasound plane, the interventional method moves to step 80 where the control circuitry determines if the interventional instrument is aligned with the target. If the interventional instrument is aligned with the target, the target may be highlighted per step 82. For example, as illustrated in FIG. 6, the out of plane navigation display 122 may include a depiction of the interventional instrument 34 appearing to head in the direction of the target 110, with the alignment indicator 116 further illustrating that the position of the interventional instrument 34 is aligned and on target.

Again returning to FIG. 2, step 82 may include providing audible feedback. For example, the ultrasound system may provide the user with a pulsed audible tone at a frequency within the normal human auditory range, such as between 85 and 255 Hz. The time between the audible tone pulses may decrease as the interventional instrument gets closer to the target, and/or, as before, the frequency or pitch of the tone may be altered. At step 84, the user may move the interventional instrument toward the target. Next, at step 86, the control circuitry may determine whether the target is reached. If the target is not reached, the method returns to step 80. Conversely, if the target is reached, the user completes the medical procedure per step 68. In addition, the ultrasound system may provide the user with an uninterrupted audible tone at a frequency within the normal human auditory range, for example.

If the interventional instrument is not aligned at step 80, an intercept point may be displayed on the navigation display per step 88. At step 90, the control circuitry may determine whether the interventional instrument is heading behind the target. If the interventional instrument is headed from behind the target in a direction toward but overshooting the target, the intercept point may be displayed as if the interventional instrument were heading ahead of the target. For example, FIG. 7 depicts the interventional instrument 34 with a distorted target 124 illustrating the location where the interventional instrument 34 would intercept the ultrasound image 100 if the interventional instrument 34 were inserted as described. The interventional instrument 34 and/or the distorted target 124 may be portrayed in any color useful to demonstrate that the interventional instrument 34 is not aligned with the target 110. Furthermore, the color of the interventional instrument 34 and/or the distorted target 124 may transition through various color shades as the interventional instrument 34 and/or distorted target 124 approach or move further away from the target 110.

Returning to FIG. 2, at step 92 the ultrasound system may provide the user with an uninterrupted audible tone above the range of normal human auditory frequencies, such as above 255 Hz. Furthermore, the frequency may adjust lower or higher as the position of the interventional instrument moves respectively closer or further away from alignment with the target. Next, the user may reposition the interventional instrument at step 94, then return to step 80 where the steps may be repeated until the target is reached and the medical procedure is complete.

Resuming the method at step 90, if the control circuitry determines that the interventional instrument is heading behind the target, a distorted target 126 may be portrayed on the navigation display representing the interventional instrument as being behind the target per step 96. For example, FIG. 8 depicts the interventional instrument 34 and distorted target 126 on the ultrasound image 100. With the distorted target 126 depicted on the navigation display 122, it may appear as if the interventional instrument 34 is heading behind the target 110.

Again returning to FIG. 2, at step 96 the ultrasound system may provide the user with an uninterrupted audible tone below the range of normal human voice frequencies when the interventional instrument is heading behind the target, such as below 85 Hz. The user may reposition the interventional instrument at step 94, then return to step 80 and may repeat the steps until the target is reached and the medical procedure is complete.

The phrases “behind the target,” “in front of the target,” and “ahead of the target” are used in the present disclosure to refer to providing a visual indication of the interventional instrument, the projected path of the interventional instrument (trajectory), and/or the distorted target or location of interception of the imaging plane (i.e., not strictly within the plane or slab). For examples of such visual indications see FIGS. 4, 5, 7, and 8, in addition to the descriptions relating to those figures. Such indications “transverse” to the imaging plane include color changes, dimensional changes, and any other indications that inform the viewer that the trajectory is moved or oriented forwardly or rearwardly with respect to the imaging plane.

It should be understood that the illustrations in FIGS. 3 through 8 are examples of certain presently contemplated ways in which ultrasound images, interventional instruments, and anatomy may be displayed. Many different variations may be devised for providing such navigational aids. Furthermore, the audible tones described are meant to be examples of how audible feedback can be used to assist an operator in performing medical procedures. It should also be noted that algorithms for determining the trajectory of an interventional instrument are generally known in the art, and any such algorithm may be used as a basis for the navigational aid displays according to the present disclosure. For example, one such technique is described in U.S. Pat. No. 6,733,458, entitled “Diagnostic Medical Ultrasound Systems and Methods Using Image Based Freehand Needle Guidance,” to Steins et al., issued on May 11, 2004, which is hereby incorporated into the present disclosure by reference.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An interventional guidance method, comprising:
generating an ultrasound image of a subject anatomy of interest;
superimposing on the ultrasound image a visual indication of at least one of projection of a position of an interventional device, trajectory of the interventional device, and a location at which the interventional device will intercept an ultrasound imaging plane; and
dynamically altering an aspect of the superimposed visual indication during an interventional procedure, including a dynamic indication of a trajectory of the interventional instrument transverse to the imaging plane or an interception location of the trajectory of the interventional instrument with the imaging plane.

2. The method of claim 1, comprising altering a visual aspect of the subject anatomy of interest to highlight the subject anatomy of interest in the ultrasound image.

3. The method of claim 1, wherein the interventional device is advanced in the imaging plane, and wherein the dynamic indication of the trajectory of the interventional device is altered.

4. The method of claim 3, wherein the dynamic indication is altered in color.

5. The method of claim 3, wherein the dynamic indication is altered in perspective.

6. The method of claim 1, wherein the interventional device is advanced from outside of the imaging plane, and wherein the dynamic indication of a location at which the interventional device will intercept an ultrasound imaging plane is altered.

7. The method of claim 6, wherein the dynamic indication is altered in color.

8. The method of claim 6, wherein the dynamic indication is altered in perspective.

9. The method of claim 1, comprising providing auditory feedback indicative of at least one of proximity of the interventional device to the subject anatomy of interest, and a degree of correctness or error of a current trajectory of the interventional device to the subject anatomy of interest.

10. The method of claim 9, wherein providing auditory feedback comprises providing at least one of a varying frequency and a varying duration of the auditory feedback.

11. An interventional guidance system, comprising:
an ultrasound system configured to generate an ultrasound image of a subject anatomy of interest; and
a display configured to show the ultrasound image and a visual indication of at least one of projection of a position of an interventional device, trajectory of the interventional device, and a location at which the interventional device will intercept an ultrasound imaging plane, the visual indication superimposed on the ultrasound image, wherein an aspect of the superimposed visual indication is dynamically altered during an interventional procedure, including a dynamic indication of a trajectory of the interventional instrument transverse to the imaging plane or an interception location of the trajectory of the interventional instrument with the imaging plane.

12. The system of claim 11, wherein the interventional device is advanced in the imaging plane, and wherein the dynamic indication of the trajectory of the interventional device is altered.

13. The system of claim 12, wherein the dynamic indication is altered in color.

14. The system of claim 12, wherein the dynamic indication is altered in perspective.

15. The system of claim 11, wherein the interventional device is advanced from outside of the imaging plane, and wherein the dynamic indication of a location at which the interventional device will intercept an ultrasound imaging plane is altered.

16. The system of claim 15, wherein the dynamic indication is altered in color.

17. The system of claim 15, wherein the dynamic indication is altered in perspective.

18. The system of claim 11, comprising a speaker configured to provide auditory feedback indicative of at least one of proximity of the interventional device to the subject anatomy of interest, and a degree of correctness or error of a current trajectory of the interventional device to the subject anatomy of interest.

19. An interventional guidance method, comprising:
generating an ultrasound image of a subject anatomy of interest;
superimposing on the ultrasound image a visual indication of at least one of projection of a position of an interventional device, trajectory of the interventional device, and a location at which the interventional device will intercept an ultrasound imaging plane; and
providing auditory feedback indicative of at least one of proximity of the interventional device to the subject anatomy of interest, and a degree of correctness or error of a current trajectory of the interventional device to the subject anatomy of interest.

20. The method of claim 19, wherein providing auditory feedback comprises providing at least one of a varying frequency and a varying duration of the auditory feedback as the interventional device approaches the subject anatomy of interest.
